(19) United States
(12) Patent Application Publication
(10) Pub. No.: US 2024/0347192 A1
(43) Pub. Date: Oct. 17, 2024

(54) SYSTEMS AND METHODS FOR REAL-TIME SYSTEMATIC HEALTH CARE

(71) Applicant: TAD HEALTH INC., Newport Beach, CA (US)

(72) Inventor: Benjamin Houston GREINER, Newport Beach, CA (US)

(21) Appl. No.: 18/511,328

(22) Filed: Nov. 16, 2023

Related U.S. Application Data
(63) Continuation of application No. PCT/US2022/029482, filed on May 16, 2022.
(60) Provisional application No. 63/189,595, filed on May 17, 2021.

(51) Int. Cl. G16H 40/67; A61B 5/00; G16H 10/60; G16H 80/00

(52) U.S. Cl. CPC G16H 40/67; A61B 5/747; A61B 5/748; G16H 10/60; G16H 80/00

(57) ABSTRACT

Disclosed herein are systems and methods for real-time systematic healthcare. The methods may comprise prompting a user to indicate the user or a subject different than the user experiencing or having a mental health crisis and providing assistance to the user or the subject. The methods may also generally involve connecting the user or the subject to emergency services or other support options in real-time as a mental health crisis is occurring.

### REFERENCE

This application is a continuation of International Application No. PCT/US2022/029482, filed on May 16, 2022, which claims priority to U.S. Provisional Application No. 63/189,595, filed on May 17, 2021, each of which is entirely incorporated herein by reference.

### BACKGROUND

A mental health crisis can be a life threatening situation in which an individual can be imminently threatening harm to himself or herself or to others, severely disorientated or out of touch with reality, has a severe inability to function, or is otherwise distraught and out of control. Individuals experiencing a mental health crisis may be in need of immediate assistance such as mental crisis resources but may have limited access thereto.

### SUMMARY

Recognized herein is a need for systems and methods for providing an immediate assistance (e.g., assistance in real-time) to an individual who is experiencing a mental health crisis or emergency. The methods and systems can comprise web-based interfaces which may facilitate establishing a connection between such individual and mental health crisis resources (e.g., in real-time) as the mental crisis or emergency is occurring.

An aspect of the present disclosure provides a computer-implemented method for providing mental crisis assistance, comprising: (a) providing a first page on a graphical user interface (GUI), wherein the first page comprises a button, wherein the button is accessible to direct a user to a second page; (b) receiving, from the user, input on the button; (c) upon receiving the input from the user in (b), directing the user to a second page on the GUI, wherein the second page prompts the user to select the user or a subject different than the user as being in need of assistance for a mental crisis; and (d) receiving from the user input on the second page, which input identifies the user or the subject as needing assistance for the mental crisis.

In some embodiments, the method further comprises providing the user or the subject treatment for the mental crisis. In some embodiments, the providing the treatment for the mental crisis comprises placing a voice-over-Internet Protocol (VOIP) call or a short message service (SMS) message to one or more emergency services. In some embodiments, the one or more emergency services comprise 911, a suicide hotline, 741-741, a mental health emergency hotline, a mental health emergency response line, or any combination thereof. In some embodiments, the VOIP call or the SMS message is directed to a telephone number associated with the one or more emergency services. In some embodiments, the telephone number is part of information regarding the plurality of emergency services stored in a database. In some embodiments, the information includes a plurality of telephone numbers associated with a plurality of emergency services, the plurality of telephone numbers comprising the telephone number. In some embodiments, the method further comprises providing a list of alternate support options. In some embodiments, the list of alternate support options includes a support system and one or more mental health care professionals. In some embodiments, the GUI comprises a web-based user interface. In some embodiments, the method further comprises prompting the user to exit the GUI. In some embodiments, the method further comprises prompting the user to confirm that the user or the subject is not in a mental health crisis prior to exiting the GUI. In some embodiments, the method further comprises providing the user with an option to restart the GUI. In some embodiments, the subject is a related to or a friend of the user. In some embodiments, the GUI is embeddable in a web site. In some embodiments, the GUI is shareable via a web link. In some embodiments, steps (a)-(d) is completed in time for emergency services to arrive as the mental crisis is occurring.

Another aspect of the present disclosure provides a method for implementing a web-based crisis response, comprising: (a) establishing a connection with a computing device; (b) via a web-based user interface: (i) prompting a user to request assistance for a mental crisis; (ii) prompting the user to select, for the user or a subject different from the user, at least one of a plurality of emergency services; and (iii) responsive to the user selecting the at least one of the plurality of emergency services, activating a software application, on or via the computing device, to place a voice-over-Internet Protocol (VOIP) call or a short message service (SMS) message to the at least one of the plurality of emergency services.

In some embodiments, the method further comprises, responsive to the user not selecting the at least one of the plurality of emergency services, providing a list of alternative support options. In some embodiments, the list of alternate support options includes a support system and one or more mental health care professionals. In some embodiments, the method further comprises prompting the user to exit the web-based user interface. In some embodiments, the method further comprises prompting the user to confirm that the user or the subject is not in a mental health crisis prior to exiting the web-based user interface. In some embodiments, the method further comprises providing the user with an option to restart the web-based interface. In some embodiments, the method further comprises: prior to (i), prompting the user to confirm that the user or the subject is or is suspected of having a mental health crisis. In some embodiments, the subject is a related to or a friend of the user. In some embodiments, the plurality of emergency services comprises 911, a suicide hotline, 741-741, a mental health emergency hotline, a mental health emergency response line, or any combination thereof. In some embodiments, the VOIP call or the SMS message is directed to a telephone number associated with the at least one of the plurality of emergency services. In some embodiments, the telephone number is part of information regarding the plurality of emergency services stored in a database. In some embodiments, the information includes a plurality of telephone numbers associated with the plurality of emergency services comprising the telephone number. In some embodiments, the prompting of (i) is performed by deploying a clickable web object. In some embodiments, the prompting of (ii) is performed by loading and presenting a landing web page to the user. In some embodiments, the prompting of (i) is performed by loading and presenting a landing web page to the user. In some embodiments, the computing device is associated with the user, and wherein the web-based user interface is presented on the computing device. In some embodiments, the computing device is not associated with the user, wherein the web-based user interface is presented on an alternative computing device associated with the user. In some embodiments, the web-based user interface is embeddable in a web site. In some embodiments, the web-based user interface is shareable via a web link. In some embodiments, steps (i)-(iii) are completed in time for emergency services to arrive as an emergency is occurring.

Another aspect of the present disclosure provides a computer-implemented method for providing mental crisis assistance, comprising: (a) providing a first page on a graphical user interface (GUI), wherein the first page comprises a button, wherein the button is accessible to direct a user to a second page; (b)receiving, from the user, input on the button; (c) upon receiving the input from the user in (b), directing the user to a second page on the GUI, wherein the second page comprises a list of services for providing mental crisis assistance determined at least in part based on a location associated with the user.

In some embodiments, the method further comprises., prior to (c), determining whether the user is associated with personalized information in a database comprising personalized information of one or more users. In some embodiments, the list of emergency medical services is further determined at least in part based on the personalized information associated with the user. In some embodiments, the method further comprises prompting the user to select the user or a subject different from the user as being in need of assistance for a mental crisis. In some embodiments, the method further comprises providing the user or the subject treatment for the mental crisis. In some embodiments, the treatment for the mental crisis comprises placing a voice-over-Internet Protocol (VOIP) call or a short message service (SMS) message to a service of the list of services. In some embodiments, the VOIP call or SMS message is directed to a telephone number associated with the service of the list of services. In some embodiments, the telephone number is part of information regarding the list of services stored in a database. In some embodiments, the information includes a plurality of telephone numbers associated with a plurality of services for providing mental crisis assistance, the plurality of telephone numbers comprising the telephone number. In some embodiments, steps (a)-(c) are completed in time for emergency services to arrive as the mental crisis is occurring. In some embodiments, the subject is a relative or a friend of the user. In some embodiments, the list of services comprises 911, 988, a suicide hotline, 741-741, a mental health emergency hotline, a mental health emergency response line, or any combination thereof. In some embodiments, the list of services comprises a hospital, an outpatient healthcare provider, one or more mental health professionals, or a peer support program. In some embodiments, the GUI comprises a web-based user interface. In some embodiments, the method further comprises prompting the user to exit the GUI. In some embodiments, the method further comprises prompting the user to confirm that the user or a subject other than the user is not in a mental health crisis prior to exiting the GUI. In some embodiments, the method further comprises providing the user with an option to restart the GUI. In some embodiments, the GUI is embeddable in a web site. In some embodiments, the GUI is shareable via a web link.

Another aspect of the present disclosure provides a method for connecting a subject with a mental health resource, comprising: (a) establishing a connection with a computing device; (b) via a web-based interface: (i) prompting a user to request mental health assistance for the user or the subject; (ii) determining a location associated with the user or the subject; and (iii) prompting the user to select at least one of a plurality of metal health resources for the subject, wherein the plurality of mental health resources is determined at least in part based on the location associated with the user or the subject. In some embodiments, the method further comprises, responsive to the user selecting at least one of the plurality of mental health resources, activating a software application, on or via the computing device, to place a voice-over-internet protocol (VOIP) call or short message service (SMS) message to the at least one of the plurality of mental health resources. In some embodiments, the at least one of the plurality of mental health resources comprises an emergency service. In some embodiments, the emergency service comprises 988, 911, a suicide hotline, 741-741, a mental health emergency hotline, or a mental health emergency response line. In some embodiments, the VOIP call or the SMS message is directed to a telephone number associated with the at least one of the plurality of emergency services. In some embodiments, the telephone number is part of information regarding the plurality of emergency services stored in a database. In some embodiments, the information includes a plurality of telephone numbers associated with the plurality of emergency services comprising the telephone number. In some embodiments, the method further comprises, prior to (iii), determining whether the user is associated with personalized information in a database comprising personalized information of one or more users. In some embodiments, the plurality of mental health resources is further determined at least in part based on the personalized information associated with the user. In some embodiments, the subject is the user. In some embodiments, the subject is a relative or a friend of the user. In some embodiments, the location associated with the user is a location of the computing device. In some embodiments, the location of the computing device is determined by satellite navigation, Wi-Fi positioning system, radio navigation, cellular telephone, AM broadcast, or FM broadcast. In some embodiments, the satellite navigation comprises the Global Positioning System (GPS). In some embodiments, the location associated with the user or the subject is determined by prompting the user to input the location. In some embodiments, the plurality of mental health resources comprises a hospital, an outpatient healthcare provider, one or more mental health professionals, or a peer support program. In some embodiments, the plurality of mental health resources in (iii) is within a cutoff distance of the location associated with the user of the subject. In some embodiments, the method comprises prompting the user to exit the web-based user interface. In some embodiments, the method further comprises prompting the user to confirm that the user or the subject is not in a mental health crisis prior to exiting the web-based user interface. In some embodiments, the method further comprises providing the user with an option to restart the web-based interface. In some embodiments, the method further comprises, prior to (i), prompting the user to confirm that the user or the subject is or is suspected of having a mental health crisis. In some embodiments, the prompting of (i) is performed by deploying a clickable web object. In some embodiments, the prompting of (iii) is performed by loading and presenting a landing web page to the user. In some embodiments, the prompting of (i) is performed by loading and presenting a landing web page to the user. In some embodiments, the computing device is associated with the user, and wherein the web-based user interface is presented on the computing device. In some embodiments, the computing device is not associated with the user, wherein the web-based user interface is presented on an alternative computing device associated with the user. In some embodiments, the web-based user interface is embeddable in a web site. In some embodiments, the web-based user interface is shareable via a web link. In some embodiments, wherein steps (i)-(iii) are completed in time for emergency services to arrive as an emergency is occurring.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a flow diagram of an example method for providing mental crisis assistance to a subject.

FIGS. 2A-2J illustrate an example method for providing mental crisis assistance to an individual.

FIG. 3 illustrates an example method of connecting a user or subject with a mental health resource

FIG. 4 illustrates an example method of connecting a user or subject with a mental health resource.

FIG. 5 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

An aspect of the present disclosure provides a computer-implemented method for providing mental crisis assistance. The method may comprise, e.g., providing a first page on a graphical user interface (GUI). The first page may comprise one or more clickable web objects such as a button, by which a user can be directed to one or more pages on the GUI different from the first page. Input on a clickable web object of the one or more clickable web objects from the user may be received and upon receipt of such input, the user may be directed to a second page on the GUI. The second page may include information which may prompt the user to identify if the user or an individual different than the user is experiencing a mental crisis or emergency. Alternatively or additionally, the second page may prompt the user to select if the user or an individual other than the user is in need for assistance (including immediate assistance) for a mental crisis or emergency. Upon the user selecting or identifying himself or herself or another individual as experiencing a mental crisis or emergency, or in need of immediate assistance for a mental crisis or emergency, the user may be directed to one or more other pages on the GUI or a webpage which may connect the user with one or more resources for providing mental crisis assistance.

In some cases, the methods may comprise providing mental-crisis assistance which may comprise providing the user or the individual experiencing a mental crisis or emergency with treatment for the mental crisis or emergency. Providing such treatment may comprise placing the user in contact with an emergency service. Information regarding an emergency service may be present on the one or more other pages on the GUI or a webpage. The emergency service may comprise the one or more assistance resources. The emergency service may comprise 911, a suicide hotline, 741-741, a mental health emergency hotline, a mental health emergency response line, or any combination thereof. The emergency service may be contacted by establishing an internet-based communication between the user and the emergency service. The Internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some instances, the internet-based communication may comprise a VOIP call or SMS message. The internet-based communication may be directed to a telephone number associated with the emergency service or a combination of emergency services. The user may further conference any other telephone number onto the internet-based communication. The telephone number as well as other telephone numbers associated with other emergency services may be held with other information regarding the emergency services in a database. The information in the database may comprise telephone numbers, email addresses, addresses, other contact information, other identifying information, and the like. The internet-based communication may be initiated upon receiving an input from the user.

The alternate support options may comprise the one or more assistance resources discussed above. The information about alternate support options may comprise a list of alternate support options. The information about alternate support options may be displayed on the GUI or a webpage. In some cases, the alternate support options may comprise a support system, one or more mental health care professionals, general mental health and wellness resources, access to an existing software application, or other resources for providing mental or emotional support. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; colleagues of the user or subject; another person; or any combination thereof.

Another aspect of the present disclosure provides the computer-implemented method for providing mental crisis assistance which may comprise prompting the user to provide other information. In one example, the user may be prompted to exit the GUI. The prompt to exit the GUI may comprise a clickable web object such as a button, image, or text. Upon receiving input from the user indicating the user wishes to exit the GUI, the user may further be prompted to confirm or to indicate consent that the user or another individual is not in need of mental crisis assistance. In another example, the user may be prompted to restart the GUI. Upon receiving input from the user indicating the user wishes to restart the GUI, the user may be directed back to the first page on the GUI or another page comprising the prompt to the user to request assistance for a mental crisis.

The GUI may comprise a web-based user interface. In some instances, the GUI is embeddable in a web page or shareable via a link. The GUI may be accessed using a computing device. In some cases, the computing device may comprise a remote computing device such as a personal computer, slate or tablet PC's (e.g., Apple® iPad, Samsung® Galaxy Tab), telephones, Smart phone (e.g., Apple® iPhone, Android-enabled device, Blackberry®), television, gaming device or console, wearable device (e.g., an activity tracker, such as a Fitbit®), or personal digital assistant. In some cases, the computing device may comprise a remote controller and the user may access the GUI by pressing a physical button.

Since rapid access to mental health resources is often key in ensuring positive outcomes for a subject experiencing a mental health crisis, in some instances, the computer-implemented method for providing mental crisis assistance may comprise providing one or more assistance resources in time for an emergency service to arrive as the subject is experiencing a mental health crisis.

Also provided herein is a method for implementing a web-based crisis response. The method may comprise, e.g., establishing a connection with a computing device. The computing device may comprise a web-based user interface which may prompt the user to request assistance for a mental crisis. The prompt may comprise a clickable web object such as a button, image, or text. Alternatively, the prompt may comprise loading and presenting a landing web page to the user. Input on the prompt may be received from the user, whereupon receiving such input the user may be directed to a second page on the web-based user interface. The second page may comprise a landing web page loaded and displayed to the user. The second page may prompt the user to select from at least one of a plurality of emergency services. The emergency service may comprise 911, a suicide hotline, 741-741, a mental health emergency hotline, a mental health emergency response line, or any combination thereof. The second page may further prompt the user to indicate if the user or an individual other than the user is in need of mental crisis assistance. Upon the user selecting at least one of the plurality of emergency services, a software application may be activated to establish an internet-based communication between the user and at least one of a plurality of emergency services. The software application may be located by the computing device or may be accessed via the computing device. The internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the internet-based communication comprises a VOIP call or SMS message to at least one of the plurality of emergency services. The internet-based communication may be directed to a telephone number associated with the emergency service or a combination of emergency services. The telephone number as well as other telephone numbers associated with other emergency services may be held with other information regarding the emergency services in a database. The information in the database may comprise telephone numbers, email addresses, addresses, other contact information, other identifying information, and the like. Upon the user not selecting at least one of the plurality of emergency services, the user may be directed to a list of alternate support options. In some cases, the alternate support options may comprise a support system, one or more mental health care professionals, general mental health and wellness resources, access to an existing software application, or other resources for providing mental or emotional support. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; a colleague of the user or subject; another person; or any combination thereof.

In some cases, the individual experiencing a mental health crisis may be the user. In some cases, the individual experiencing a mental health crisis may comprise an individual other than the user. The individual other than a user may comprise a friend of the user, a relative of the user, a colleague of the user, or the like.

In some cases, the method for implementing a web-based crisis response comprises prompting the user to provide other information. In one example, the user may be prompted to exit the web-based user interface. The prompt to exit the web-based user interface may comprise a clickable web object such as a button, image, or text. Upon receiving input from the user indicating the user wishes to exit the web-based interface, the user may further be prompted to confirm or to indicate consent that the user or another individual is not in need of mental crisis assistance. In another example, the user may be prompted to restart the web-based user interface. Upon receiving input from the user indicating the user wishes to restart the web-based user interface, the user may be directed back to the page comprising the prompt to the user to request assistance for a mental crisis.

The method for implementing a web-based crisis response may comprise prompting the user to confirm that the user or another individual is suspected of having or experiencing a mental health crisis. The prompt to confirm that the user or another individual is suspect of having or experiencing a mental health crisis may comprise loading and presenting a landing web page to the user.

In still another aspect of the present disclosure, the computing device may be associated with the user or may not be associated with the user. The web-based user interface may be present on the computing device or it may be present on an alternative computing device not associated with the user. In some cases, the computing device may comprise a remote computing device such as a personal computer, slate or tablet PC's (e.g., Apple® iPad, Samsung® Galaxy Tab), telephones, Smart phone (e.g., Apple® iPhone, Android-enabled device, Blackberry®), television, gaming device or console, wearable device (e.g., an activity tracker, such as a Fitbit®), or personal digital assistant. In some cases, the computing device may comprise a remote controller and the user may access the GUI by pressing a physical button.

Since rapid access to mental health resources is often key in ensuring positive outcomes for a subject experiencing a mental health crisis, in some cases, the method for implementing a web-based crisis response may comprise directing the user to one or more assistance resources in time for an emergency service to arrive as the subject is experiencing a mental health crisis.

Another aspect of the present disclosure provides a computer-implemented method for providing mental crisis assistance. The method may comprise, e.g., providing a first page on a graphical user interface (GUI). The first page may comprise one or more clickable web objects such as a button, by which a user can be directed to one or more pages on the GUI different from the first page. A second page may comprise a list of services such as those disclosed herein for providing mental crisis assistance based at least in part on a location associated with a user. The location associated with the user may be provided by a prompt given to the user on the GUI or may be determined from the device with the GUI (e.g., by GPS).

The location associated with the user may be determined by prompting the user to input a specific location or by determining a location associated with the computing device the user used to access the GUI. The location input by the user may be in the form of an address or part thereof (e.g., postal code), a reference to a landmark, geographical coordinates (e.g., latitude and longitude), or any combination thereof. Alternatively, or additionally, the location input may be based on a user positioning a pin on a map. The location of the computing device may be determined by satellite navigation (e.g., Global Positioning System), radio navigation, cellular telephone, Wi-Fi positioning system (WPS), AM broadcast, FM broadcast, or any combination thereof.

The list of services presented to the user may be based at least in part on the location associated with the user. In an example, mental crisis assistance resources are presented that fall within a cutoff distance of the location associated with the user. In some cases, mental crisis assistance resources that fall within a distance of at least about 1, 2, 3, 4, 5, 6, 7, 8, 9, 10, 11, 12, 13, 14, 15, 16, 17, 18, 19, 20, 50, 100 or more miles of the location associated with the user are presented. In some cases, mental assistance resources that fall within no more than about 100, 50, 20, 19, 18, 17, 16, 15, 14, 13, 12, 11, 10, 9, 8, 7, 6, 5, 4, 3, 2, 1, or fewer miles of the location associated with the user are presented. In some cases, the cutoff distance falls between any two of these numbers. In some cases, the user may specify the cutoff distance or a range of distances (e.g., from about 2 to about 10 miles) in which they are seeking mental health resources. In such cases, a list of mental health resources may be stored in one or more directories.

In some cases, the list of resources presented to the user may be based at least in part on personalized information associated with the user. Such personalized information may be present in a database containing information associated with one or more users. The information may comprise biographical or health data associated with the users, such as age, sex or gender, race or ethnicity, educational level, employment history, lifestyle, tastes or preferences, residence, weight, height, body mass index (BMI), known diseases or disorders, health history (e.g., past diagnoses), mental clarity, fatigue levels, mood, obsessive compulsive traits, anxiety level, stress level, neuroticism, empathy, feelings of worry or guilt, postnatal depression, response to environmental stress, and thoughts of suicide. The information may comprise data indicative of a prior use by the user of the methods and systems described herein to access one or more mental health resources. The personalized information may be used to determine the list of mental health resources presented to the user, such as by selecting resources appropriate to the user's documented mental health history or prior use of mental health resources.

In some cases, the methods may comprise providing mental-crisis assistance which may comprise providing the user or the individual experiencing a mental crisis or emergency with treatment for the mental crisis or emergency. Providing such treatment may comprise placing the user in contact with an emergency service. Information regarding an emergency service may be present on the one or more other pages on the GUI or a webpage. The emergency service may comprise the one or more assistance resources. The emergency service may comprise 988, 911, another emergency telephone number (e.g., associated with a specific locale, such as a certain country), a suicide hotline, 741-741, a mental health emergency hotline, a mental health emergency response line, or any combination thereof. The emergency service may be contacted by establishing an internet-based communication between the user and the emergency service. The Internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some instances, the internet-based communication may comprise a VOIP call or SMS message. The internet-based communication may be directed to a telephone number associated with the emergency service or a combination of emergency services. The user may further conference any other telephone number onto the internet-based communication. The telephone number as well as other telephone numbers associated with other emergency services may be held with other information regarding the emergency services in a database. The information in the database may comprise telephone numbers, email addresses, addresses, other contact information, other identifying information, and the like. The internet-based communication may be initiated upon receiving an input from the user.

In some cases, the methods may comprise providing the user or subject with information about one or more mental health resources. The mental health resources may comprise one or more alternate support options discussed above. The information about alternate support options may comprise a list of alternate support options. The information about alternate support options may be displayed on the GUI or a webpage. In some cases, the alternate support options may comprise a support system, one or more mental health care professionals, a hospital, an outpatient healthcare provider, a peer support program, general mental health and wellness resources, access to an existing software application, or other resources for providing mental or emotional support. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; colleagues of the user or subject; another person; or any combination thereof.

In another aspect, the present disclosure provides for a web-based method of connecting a subject with a mental health resource. The method may comprise, e.g., establishing a connection with the computing device. The computing device may comprise a web-based user interface which may prompt the user to request mental health assistance. The mental health assistance may be requested for the user or for a subject other than the user. The prompt may comprise a clickable web object such as a button, image, or test. Alternatively, the prompt may comprise loading and presenting a landing web page to the user. Input on the prompt may be received from the user. The method may further comprise determining a location associated with the user or the subject. The location of the user or the subject may be provided by the prompt given to the user, another prompt to the user, or from the computing device (e.g., by GPS). Upon receiving the location data associated with the user or device, user may be prompted to select from at least one of a plurality of mental health resources. The plurality of mental health resources displayed to the user may be based at least in part on the location data associated with the user.

In an example, mental health resources are presented that fall within a cutoff distance of the location associated with the user. In some cases, mental crisis assistance resources that fall within a distance of at least about 1, 2, 3, 4, 5, 6, 7, 8, 9, 10, 11, 12, 13, 14, 15, 16, 17, 18, 19, 20, 50, 100 or more miles of the location associated with the user are presented. In some cases, mental assistance resources that fall within no more than about 100, 50, 20, 19, 18, 17, 16, 15, 14, 13, 12, 11, 10, 9, 8, 7, 6, 5, 4, 3, 2, 1, or fewer miles of the location associated with the user are presented. In some cases, the cutoff distance falls between any two of these numbers. In some cases, the user may specify the cutoff distance or a range of distances (e.g., from about 2 to about 10 miles) in which they are seeking mental health resources. In such cases, a list of mental health resources may be stored in one or more directories.

In some cases, the list of resources presented to the user may be based at least in part on personalized information associated with the user. Such personalized information may be present in a database containing information associated with one or more users. The information may comprise biographical or health data associated with the users, such as age, sex or gender, race or ethnicity, educational level, employment history, lifestyle, tastes or preferences, residence, weight, height, body mass index (BMI), known diseases or disorders, health history (e.g., past diagnoses), mental clarity, fatigue levels, mood, obsessive compulsive traits, anxiety level, stress level, neuroticism, empathy, feelings of worry or guilt, postnatal depression, response to environmental stress, and thoughts of suicide. The information may comprise data indicative of a prior use by the user of the methods and systems described herein to access one or more mental health resources. The personalized information may be used to determine the list of mental health resources presented to the user, such as by selecting resources appropriate to the user's documented mental health history or prior use of mental health resources.

In some cases, the mental health resources may comprise an emergency service. The emergency service may comprise 988, 911, another emergency telephone number (e.g., associated with a certain locale, such as a specific country), a suicide hotline, 741-741, a mental health emergency hotline, a mental health emergency response line, or any combination thereof. Upon the user selecting at least one of the plurality of emergency services, a software application may be activated to establish an internet-based communication between the user and at least one of a plurality of emergency services. The software application may be located by the computing device or may be accessed via the computing device. The internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the internet-based communication comprises a VOIP call or SMS message to at least one of the plurality of emergency services. The internet-based communication may be directed to a telephone number associated with the emergency service or a combination of emergency services. The telephone number as well as other telephone numbers associated with other emergency services may be held with other information regarding the emergency services in a database. The information in the database may comprise telephone numbers, email addresses, addresses, other contact information, other identifying information, and the like.

Alternatively or additionally, the list of mental health resources may comprise one or more alternate support options. In some cases, the alternate support options may comprise a hospital, an outpatient healthcare facility, a support system, one or more mental health care professionals, general mental health and wellness resources, a peer support network, access to an existing software application, or other resources for providing mental or emotional support. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; a colleague of the user or subject; another person; or any combination thereof.

In some cases, the subjecting connected with the mental health resource may be the user. In some cases, the subject seeking the mental health resource may comprise an individual other than the user. The individual other than a user may comprise a friend of the user, a relative of the user, a colleague of the user, or the like.

In some cases, web-based method for connecting the subject with a mental health resource comprises prompting the user to provide other information. In one example, the user may be prompted to exit the web-based user interface. The prompt to exit the web-based user interface may comprise a clickable web object such as a button, image, or text. Upon receiving input from the user indicating the user wishes to exit the web-based interface, the user may further be prompted to confirm or to indicate consent that the user or another individual is not in need of mental crisis assistance. In another example, the user may be prompted to restart the web-based user interface. Upon receiving input from the user indicating the user wishes to restart the web-based user interface, the user may be directed back to the page comprising the prompt to the user to request assistance for a mental crisis.

In still another aspect of the present disclosure, the computing device may be associated with the user or may not be associated with the user. The web-based user interface may be present on the computing device or it may be present on an alternative computing device not associated with the user. In some cases, the computing device may comprise a remote computing device such as a personal computer, slate or tablet PC's (e.g., Apple® iPad, Samsung® Galaxy Tab), telephones, Smart phone (e.g., Apple® iPhone, Android-enabled device, Blackberry®), television, gaming device or console, wearable device (e.g., an activity tracker, such as a Fitbit®), or personal digital assistant. In some cases, the computing device may comprise a remote controller and the user may access the GUI by pressing a physical button.

Since rapid access to mental health resources is often key in ensuring positive outcomes for a subject experiencing a mental health crisis, in some cases, the method for implementing a web-based crisis response may comprise directing the user to one or more assistance resources in time for an emergency service to arrive as the subject is experiencing a mental health crisis.

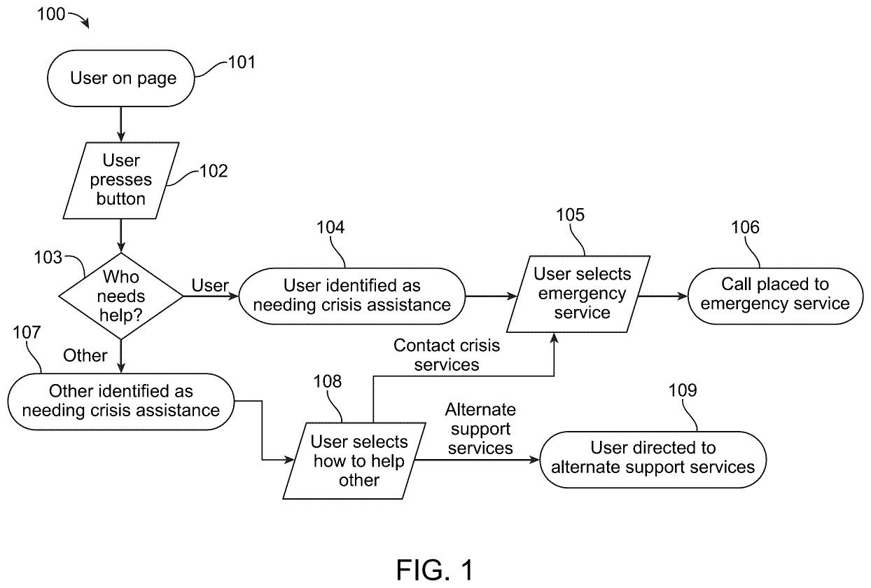

FIG. 1 is a flow diagram illustrating an example method for providing mental crisis assistance 100 to a subject, in accordance with an aspect of the present disclosure. In step 101, the user is prompted to give input on a first page provided to the user on a graphical user interface (GUI). In step 102, the user is provided a first page on the GUI which when clicked by the user directs the user to a second page. In some cases, the GUI may be embeddable in a web site, shareable via a web link, or some combination thereof. Once on the second page, the user is directed in step 103 to indicate whether the assistance is sought for the user or for another subject. If the user indicates the user is the subject in need of assistance, step 104 identifies the user as the subject in need of assistance. Alternatively, if the user indicates another is the subject in need of assistance, step 107 identifies another as the subject in need of assistance. The subject in need of assistance may comprise the user, a relative of the user, a friend of the user, a colleague of the user, or the like.

Once the subject in need of assistance has been identified, the method may then continue to provide such assistance, in some cases comprising treatment or connection with resources to provide treatment for the mental crisis. Such treatment may, among other things, comprise connecting the user with one or more emergency service. If the subject in need of assistance has been identified as the user, step 105 prompts the user to contact one or more emergency services. Upon doing so, the user is connected in step 106 the one or more emergency services. By way of nonlimiting example, the one or more emergency services may comprise 988, 911, another emergency telephone number (e.g., one associated with a particular location, such as a certain country), a suicide hotline, 741-741, a mental health emergency hotline a mental health emergency response line, or the like. In some cases, information of the one or more emergency services is stored in a database. The information in the database may comprise telephone numbers, email addresses, addresses, other contact information, other identifying information, and the like. The user may be connected to the one or more emergency services through an internet-based communication. By way of nonlimiting example, the internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the step of providing the user with some an internet-based communication may comprise activating a software application comprising, by way of nonlimiting example, an app or a web browser. In some cases, the internet-based communication comprises a VOIP call or SMS message. In some cases, the VOIP call or SMS may be directed to a telephone number associated with the one or more emergency services.

If the subject in need of assistance has been identified as another, step 108 prompts the user to choose from the options of contacting an emergency service or viewing information about alternate support options. If the user selects to contact emergency services, the user is directed in step 105 to contact one or more emergency services. Upon doing so, the user is connected in step 106 the one or more emergency services. By way of nonlimiting example, the one or more emergency services may comprise 911, a suicide hotline, 741-741, a mental health emergency hotline a mental health emergency response line, or the like. In some cases, information the one or more emergency services are stored in a database. The information in the database may comprise telephone numbers, email addresses, addresses, other contact information, other identifying information, and the like. The one or more emergency services may be contacted by establishing an internet-based communication between the one or more emergency services and the user. By way of nonlimiting example, the internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the step of providing the user with an internet-based communication may comprise activating a software application including, by way of nonlimiting example, an app or a web browser. In some cases, the internet-based communication comprises a VOIP call or SMS message. In some cases, the VOIP call or SMS message may be directed to a telephone number associated with the one or more emergency services. In some cases, the user may at step 110 add another user in real-time to the call placed to emergency services. By way of nonlimiting example, the user may add another user by sharing a link, conferencing in another telephone number, or the like. In some cases, the link may be shared by wireless communication such as SMS or MMS. Alternatively or additionally, the user may select at step 108 to be connected to information about alternative support options. The alternate support options may comprise, by way of nonlimiting example, a support system, options to connect with one or more healthcare professionals, general mental health and wellness resources, or other resources for providing mental or emotional support. In some cases, the alternate support options may comprise a support system or one or more healthcare professionals. Upon selecting an alternate support option, the user is directed in step 109 to the alternate support option or information about the alternate support option.

In some cases, the method further comprises giving the user the option at any time during the method to exit the GUI. In some cases, the user may be prompted to confirm that the user or another subject is not in a mental health crisis before exiting.

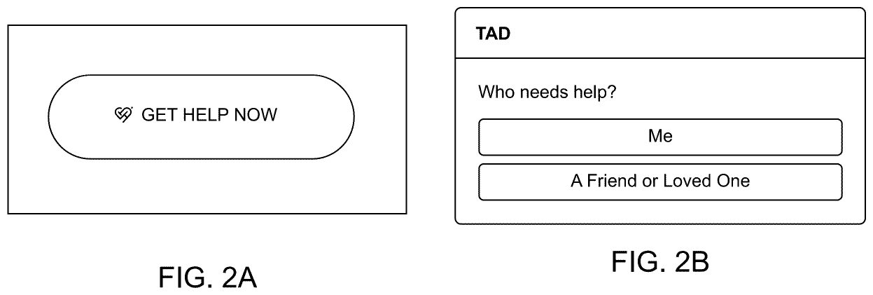

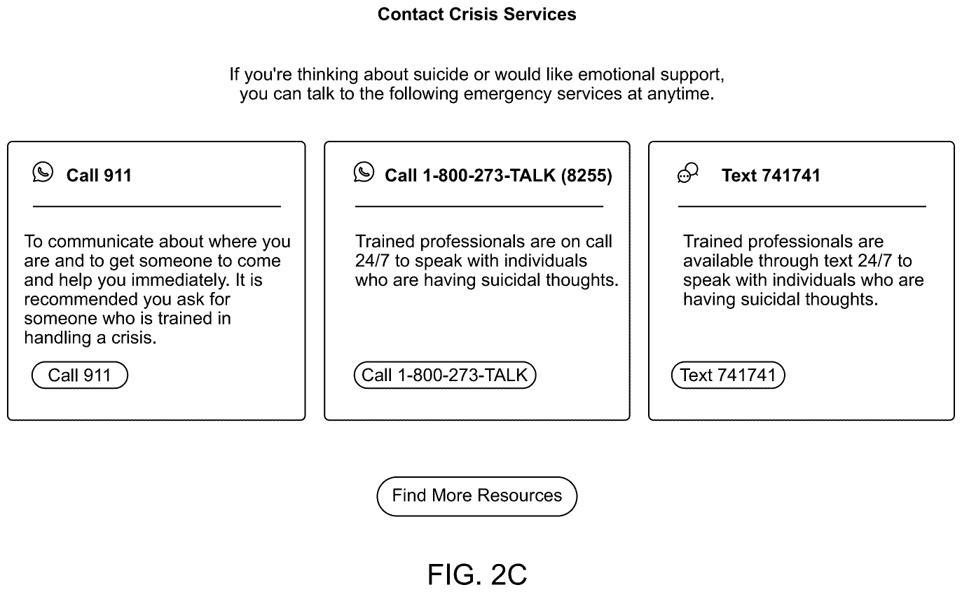

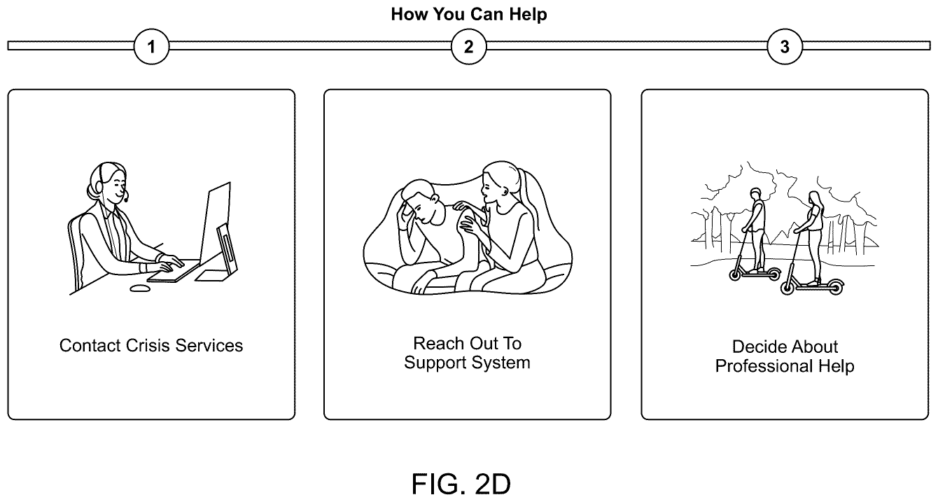

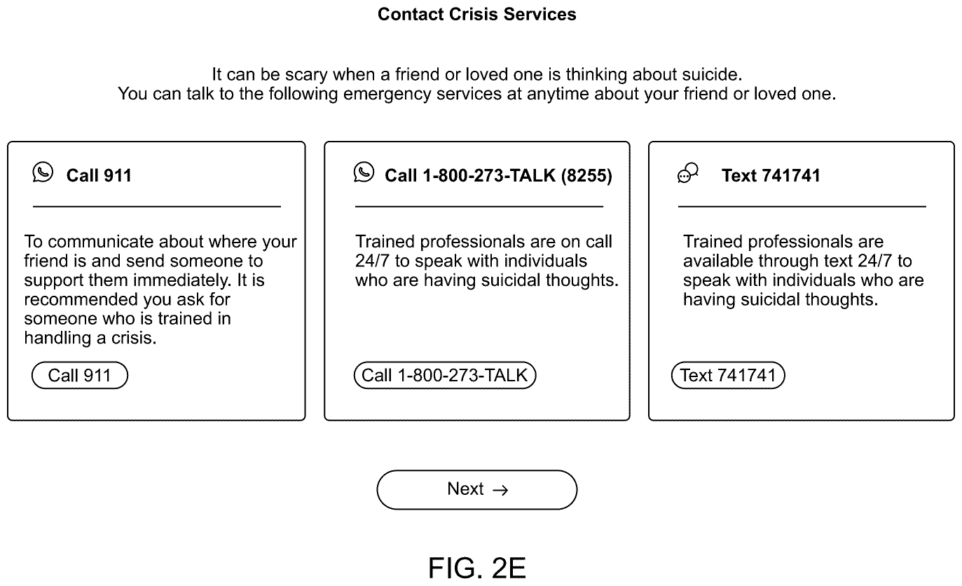

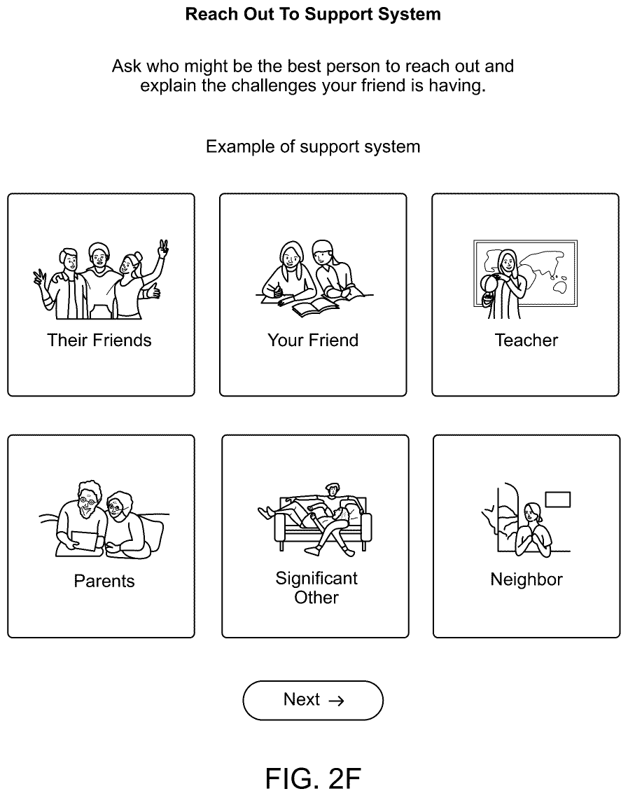

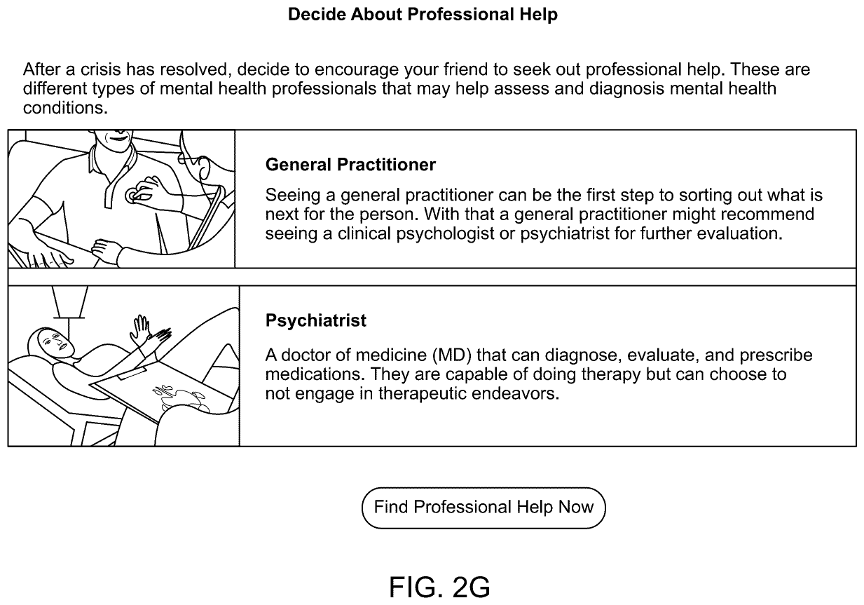

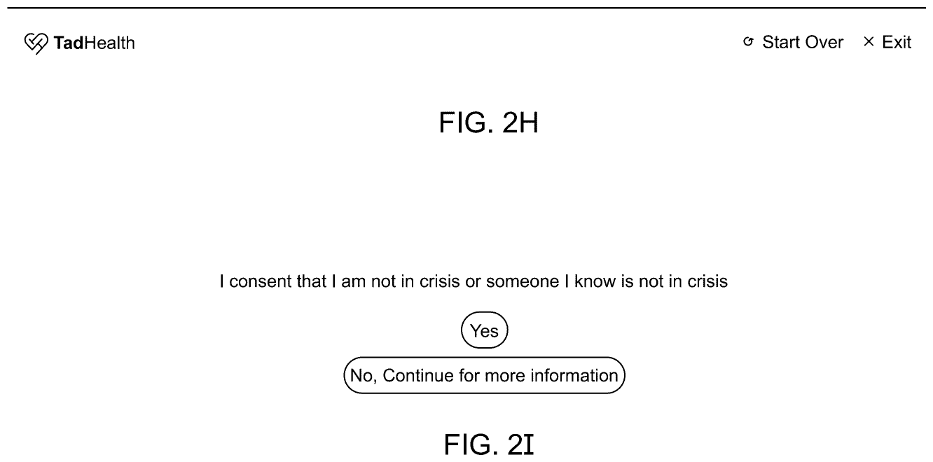

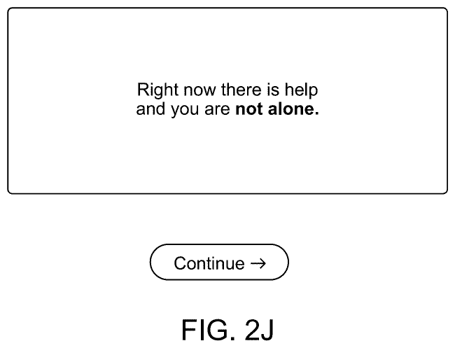

FIGS. 2A-2J illustrate providing mental crisis assistance in one example of the present disclosure. FIG. 2A illustrates a prompt to a user to request assistance for a mental crisis. By way of nonlimiting example, the prompt may comprise a clickable web object comprising text or an image, a button or more than one button, or the like. In some cases, the prompt comprises a clickable web object comprising a button. When the user selects the prompt, the invention directs the user to another prompt to indicate the subject in need of mental crisis assistance. The prompt may be on another page on the GUI or a web page. The prompt may comprise an additional button.

FIG. 2B shows an example prompt to the user to indicate whether the subject in need of mental crisis assistance is the subject or another. In some cases, the prompt comprises presenting a landing web page to the user. The user may indicate the subject in need of mental crisis assistance by selecting the appropriate choice from the prompt. Once the user has indicated the subject in need of mental crisis assistance, the user is then directed to another web page which prompts the user to seek mental crisis assistance for the subject.

FIG. 2C shows an example prompt to the user to select one or more from a plurality of emergency services. As shown in FIG. 2C, the plurality of emergency services may comprise 911, a suicide hotline, and a crisis line 741-741. By way of nonlimiting example, the one or more emergency services may comprise 911, a suicide hotline, 741-741, a mental health emergency hotline a mental health emergency response line, or the like. In some cases, information the one or more emergency services are stored in a database. The information in the database may comprise telephone numbers, email addresses, addresses, other contact information, other identifying information, and the like. Once the user selects at least one of the plurality of emergency services from the prompt, the one or more emergency services may be contacted by established an internet-based communication between the one or more emergency services and the user. By way of nonlimiting example, the internet-based communication may comprise instant messaging (IM), internet telephony or voice-over-Internet Protocol (VOIP) call, email, internet relay chat (IRC), videoconferencing, or wireless communications including short message service (SMS) and multimedia message service (MMS). In some cases, the step of providing the user with an internet-based communication may comprise activating a software application including, for example, an app or a web browser. In some cases, the internet-based communication comprises a VOIP call or SMS. In some cases, the VOIP call or SMS may be directed to a telephone number associated with the one or more emergency services.

FIG. 2D shows an example prompt which is displayed when the user indicates the subject in need of mental crisis assistance is another. The subject may comprise a relative of the user, a friend of the user, a colleague of the user, or the like. The prompt indicates to the user a way or a plurality of ways in which the user may provide mental crisis assistance to the subject in need thereof. By way of nonlimiting example, the way or plurality of ways in which the user may provide crisis assistance to the subject in need thereof may comprise contacting emergency services, a support system, alternate support options, or some combination thereof. The alternate support options may comprise, by way of nonlimiting example, a support system, options to connect with one or more healthcare professionals, general mental health and wellness resources, or other resources for providing mental or emotional support. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; one or more users in a database; another person; or any combination thereof.

In an example, the prompt which is displayed when the user indicated the subject in need of mental crisis assistance is another may prompt the user to navigate to another window such as that depicted in FIG. 2E, which shows an example prompt to the user to select one or more from a plurality of emergency services. As shown in FIG. 2E, the plurality of emergency services may comprise 911, a suicide hotline, and a crisis line 741-741. By way of nonlimiting example, the one or more emergency services may comprise 911, a suicide hotline, 741-741, a mental health emergency hotline a mental health emergency response line, or the like. In some cases, information the one or more emergency services are stored in a database. The information in the database may comprise telephone numbers, email addresses, addresses, other contact information, other identifying information, and the like. Once the user selects at least one of the plurality of emergency services from the prompt, the one or more emergency services may be contacted by providing the user with an internet-based communication. By way of nonlimiting example, the internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the step of providing the user with an internet-based communication may comprise activating a software application including, for example, an app or a web browser. In some cases, the internet-based communication comprises a VOIP call or SMS message. In some cases, the VOIP call or SMS may be directed to a telephone number associated with the one or more emergency services.

In an example, the user may next be prompted to navigate to another page as depicted in FIG. 2F which directs the user to reach out to the support system of the subject in need of mental crisis assistance. The support system may comprise individuals of trust or with particular expertise or experience in providing mental or emotional support in the life of the subject in need of mental crisis assistance. The support system may comprise the user's friends; the subject's friends, if the subject is different from the user; a teacher; parents; a significant other; a colleague; one or more users in a database; another person; or any combination thereof.

In an example, the user may next be prompted to navigate to another page as depicted in FIG. 2G which displays information about one or more mental health care professionals.

In an example, at any point while seeking mental crisis assistance, the user may be prompted to exit, for example by pressing an appropriate button. FIG. 2H illustrates a prompt to the user to exit. Upon doing so, the user is prompted with a window asking the user to confirm that the user of another is not in a mental health crisis. FIG. 2I depicts an example of such a window. In an example, the window may prompt the user to respond affirmatively or negatively. If the user responds affirmatively, the user exits the GUI, such as by being directed back to the page that contained the prompt to the user to request assistance for a mental crisis. If the user responds negatively, the user is directed back to a listing of support services for a subject experiencing a mental crisis.

In an example, a prompt such as that depicted in FIG. 2J may appear at any time to display an encouraging statement to the user.

In an example, at any time a user may be prompted to click a button to start over. Upon doing so, the user is directed back to the prompt to indicate the subject in need of mental crisis assistance such as that depicted in FIG. 2B.

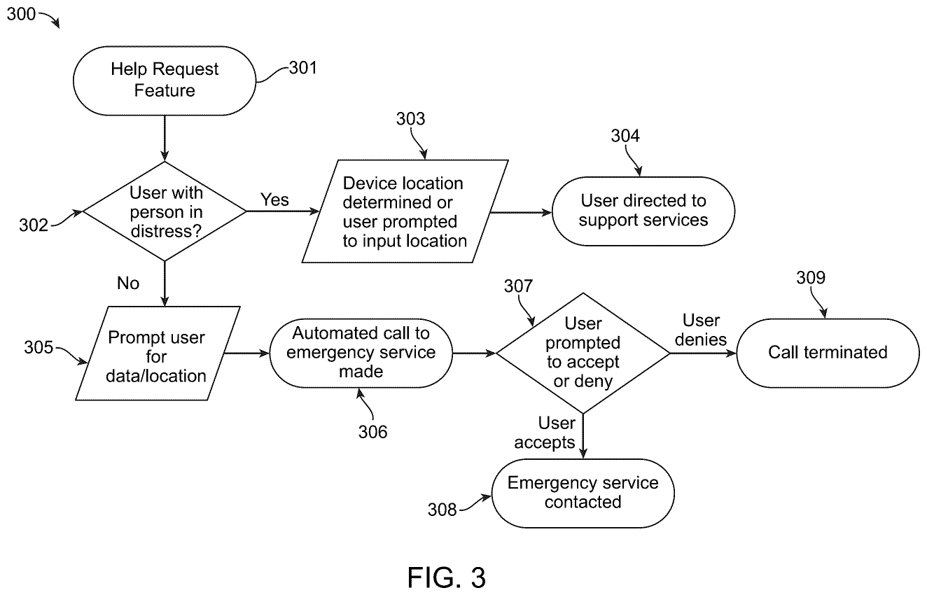

FIG. 3 is a flow diagram illustrating an example method 300 for connecting a user or subject with a mental health resource. In step 301, the user interacts with a help request feature, e.g., by giving input on a first page (such as button) on a graphical user interface (GUI). The GUI may be embeddable in a web site, shareable via a web link, or some combination thereof. Alternatively, the user may have already indicated a request for help (e.g., in step 102 of FIG. 1) and is now directed to one or more mental health resources, such as a list of alternate support services (e.g., in step 108 of FIG. 1). In the step 302, the user is prompted to indicate whether assistance is sought for the user or another subject (e.g., person in distress). The prompt in step 302 may be on a second page which the user is directed to upon interacting with the prompt in step 301. If the user indicates that help is sought for the user (e.g., the user is the subject), a location may be determined for the user in step 303. The location may be determined from a location of the device by which the user has accessed the help request feature. The location of the device may be determined by satellite navigation (e.g., Global Positioning System), radio navigation, cellular telephone, Wi-Fi positioning (WPS), AM broadcast, FM broadcast, or any combination thereof. Upon providing a location, the user may be directed (e.g., on a third webpage) to a list of mental health resources in step 304. The list may be based on the location associated with the user from step 303. Alternatively, or additionally, the list of resources may be based in part on personalized information associated with the user. The personalized information may be stored in one or more databases or supplied by the user (e.g., in response to a prompt). The personalized information may include biographical or health data such as age, sex or gender, race or ethnicity, educational level, employment history, lifestyle, tastes or preferences, residence, weight, height, body mass index (BMI), known diseases or disorders, health history (e.g., past diagnoses), mental clarity, fatigue levels, mood, obsessive compulsive traits, anxiety level, stress level, neuroticism, empathy, feelings of worry or guilt, postnatal depression, response to environmental stress, and thoughts of suicide. The information may comprise data indicative of prior use by the user of the methods and system described herein to access one or more mental health resources. The list of resources may include emergency resources, such as 988, 911, another emergency telephone number (e.g., one associated with a particular location, such as a certain country), a suicide hotline, 741-741, a mental health emergency hotline, or a mental health emergency response line; non-emergency services such as a hospital, an outpatient healthcare provider, one or more mental health professionals, or a peer support program; or any combination thereof. In some cases, information of the one or more mental health services is stored in a database. The information in the database may comprise telephone numbers, email addresses, addresses, other contact information, other identifying information, and the like. The user may be connected to the one or more services through an internet-based communication. By way of nonlimiting example, the internet-based communication may comprise instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the methods may comprise activating a software application comprising, by way of nonlimiting example, an app or a web browser. In some cases, the internet-based communication comprises a VOIP call or SMS message. In some cases, the VOIP call or SMS may be directed to a telephone number associated with the one or more resources.

Alternatively, if the user indicated at step 302 that assistance is sought for an individual other than the user, the user may be prompted at step 305 to provide a location associated with the subject. The user may provide the location associated with the subject in textual form, such as an address or part thereof, a reference to a landmark, geographical coordinates, or any combination thereof. Alternatively or additionally, the user may provide the location associated with the subject by interacting with the GUI, such as by positioning a pin on a map. Upon indicating a location, an emergency service may be contacted in step 306. The contact may be through an internet-based communication such as a instant messaging (IM), internet telephony or voice-over-Internet Protocol (VOIP) call, email, internet relay chat (IRC), videoconferencing, or wireless communications including short message service (SMS) message and multimedia message service (MMS) message. In some cases, the step of providing the user with an internet-based communication may comprise activating a software application including, by way of nonlimiting example, an app or a web browser. In some cases, the internet-based communication comprises a VOIP call or SMS message. In some cases, the VOIP call or SMS message may be directed to a telephone number associated with the one or more emergency services. After placing the call, the user may be prompted in step 307 to accept or deny the call to emergency services. If the user accepts, the user or the subject may be connected with the emergency service in step 308. Alternatively, if the user rejects the call, the call may be terminated, as illustrated in step 309.

After step 303 or 305 (e.g., a step soliciting or acquiring location data associated with the user or subject), an optional authentication or verification step may take place to confirm the user or subject wishes to proceed with connection to a mental health resource or to ensure that the correct subject is being connected to a mental health resource. Authentication or authorization may take the form of a communication (such as a call, VOIP call, email, MMS, or SMS) sent to the user or subject asking for verification or confirmation of the subject to be connected with a mental health resource or for the user or subject to provide authorization for being connected with a mental health resource. In some cases, the authorization or authentication may take the form of two-factor authentication (TFA) using a device associated with the user or subject. The TFA may be provided by a third-party authentication service integrated into an application as disclosed herein. The TFA may comprise placing a communication (e.g., call, email, SMS, MMS, VOIP call) to a device associated with the subject or user.

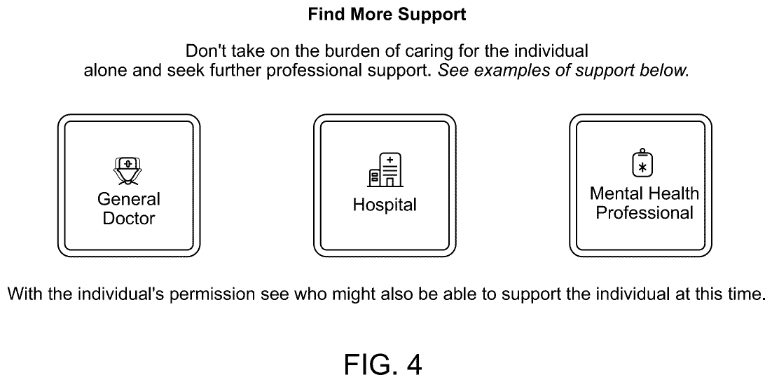

FIG. 4 illustrate connecting a user with a mental health resource in one example of the present disclosure. As illustrated in FIG. 4, the user may be prompted to select from a list of one or more mental health resources for the user or a subject other than the user. The prompt may be accessed by, e.g., prompting the user on a web-based interface to request mental health assistance for the user or a subject other than the user. Additionally, a location associated with the user or subject may be used to determine which resource or resources are listed. In an example, the user is prompted to supply a location, or a location of the device is determined (e.g., by GPS). Based on the location, a directory of one or more resources is accessed to determine which resource or resources to present to the user.

### Computer Systems

The present disclosure provides computer systems that are programmed to implement methods of the disclosure. FIG. 5 shows a computer system 501 that is programmed or otherwise configured to provide mental crisis assistance to an individual in need thereof. The computer system 501 can regulate various aspects of providing mental crisis assistance of the present disclosure, such as, for example, prompting the user to seek mental crisis assistance or providing connections to such assistance. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 550 with the aid of the communication interface 520. The network 550 can be the Internet, an internet and/or extranet, or an intranet and/or extranet that is in communication with the Internet. The network 550 in some cases is a telecommunication and/or data network. The network 550 can include one or more computer servers, which can enable distributed computing, such as cloud computing. The network 550, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 550. For instance, the computer system 501 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 550.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 555 that comprises a user interface (UI) 540 for providing, for example, a method of providing mental crisis assistance. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, prompt a user to seek mental crisis assistance for a subject in need thereof, connect a user with mental crisis assistance or prompt an additional user to join a communication with mental crisis assistance.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer-implemented method for providing mental crisis assistance, comprising:
(a) providing a first page on a graphical user interface (GUI), wherein said first page comprises a button, wherein said button is accessible to direct a user to a second page;
(b) receiving, from said user, input on said button;
(c) upon receiving said input from said user in (b), directing said user to a second page on said GUI, wherein said second page prompts said user to select said user or a subject different than said user as being in need of assistance for a mental crisis; and
(d) receiving from said user input on said second page, which input identifies said user or said subject as needing assistance for said mental crisis.

2. The method of claim 1, further comprising providing said user or said subject treatment for said mental crisis.

3. The method of claim 2, wherein said providing said treatment for said mental crisis comprises placing a voice-over-Internet Protocol (VOIP) call or a short message service (SMS) message to one or more emergency services.

4. The method of claim 3, wherein said one or more emergency services comprise 911, a suicide hotline, 741-741, a mental health emergency hotline, a mental health emergency response line, or any combination thereof.

5. (canceled)

6. (canceled)

7. (canceled)

8. The method of claim 1, further comprising providing a list of alternate support options.

9. The method of claim 8, wherein said list of alternate support options includes a support system and one or more mental health care professionals.

10. The method of claim 1, wherein said GUI comprises a web-based user interface.

11. (canceled)

12. The method of claim 1, further comprising prompting said user to confirm that said user or said subject is not in a mental health crisis prior to exiting said GUI.

13-16. (canceled)

17. The method of claim 1, wherein steps (a)-(d) are completed in time for emergency services to arrive as said mental crisis is occurring.

18.-37. (canceled)

38. A computer-implemented method for providing mental crisis assistance, comprising:
(a) providing a first page on a graphical user interface (GUI), wherein said first page comprises a button, wherein said button is accessible to direct a user to a second page;
(b) receiving, from said user, input on said button;
(c) upon receiving said input from said user in (b), directing said user to a second page on said GUI, wherein said second page comprises a list of services for providing mental crisis assistance determined at least in part based on a location associated with said user.

39. The method of claim 38, further comprising, prior to (c), determining whether said user is associated with personalized information in a database comprising personalized information of one or more users.

40. The method of claim 39, wherein said list of emergency medical services is further determined at least in part based on said personalized information associated with said user.

41. The method of claim 38, further comprising prompting said user to select said user or a subject different from said user as being in need of assistance for a mental crisis.

42. The method of claim 41, further comprising providing said user or said subject treatment for said mental crisis.

43. The method of claim 42, wherein said treatment for said mental crisis comprises placing a voice-over-Internet Protocol (VOIP) call or a short message service (SMS) message to a service of said list of services.

44.-46. (canceled)

47. The method of claim 38, wherein steps (a)-(c) are completed in time for emergency services to arrive as said mental crisis is occurring.

48. (canceled)

49. The method of claim 38, wherein said list of services comprises 911, 988, a suicide hotline, 741-741, a mental health emergency hotline, a mental health emergency response line, or any combination thereof.

50. The method of claim 38, wherein said list of services comprises a hospital, an outpatient healthcare provider, one or more mental health professionals, or a peer support program.

51. The method of claim 38, said GUI comprises a web-based user interface.

52. (canceled)

53. The method of claim 38, further comprising prompting said user to confirm that said user or a subject other than said user is not in a mental health crisis prior to exiting said GUI.

54.-85. (canceled)
